(19) United States
(12) Patent Application Publication
(10) Pub. No.: US 2021/0299313 A1
(43) Pub. Date: Sep. 30, 2021

(54) COMBUSTION DEVICE ADAPTED TO THE DIFFUSION OF FRAGRANCE

(71) Applicant: Pro-Iroda Industries, Inc., Taichung (TW)

(72) Inventor: WEI CHENG WU, Taichung (TW)

(21) Appl. No.: 17/159,697

(22) Filed: Jan. 27, 2021

(30) Foreign Application Priority Data
Mar. 31, 2020 (CN) 202020451066.3

(51) Int. Cl. A61L 9/03

(52) U.S. Cl. CPC A61L 9/037

(57) ABSTRACT

A combustion device adapted to the diffusion of fragrance has a main body which has an internal space divided into a heating zone and a fuel zone and defines a ventilation hole. The heating zone is adjacent to the top of the main body. The fuel zone is adjacent to the bottom of the main body. The fuel zone receives a wick. The ventilation hole extends vertically to the heating zone. The combustion device also includes a fragrance diffuser diffusing fragrance disposed in the heating zone. The fragrance diffuser includes an insulator and a fragrance holder housed by the insulator. The insulator defines a hollow above the wick and a groove. The fragrance holder is disposed in the groove.

### BACKGROUND OF THE INVENTION

### 1. Field of the Invention

The present invention relates to a combustion device and, particularly, to a combustion device adapted to the diffusion of fragrance.

### 2. Description of the Related Art

Taiwan Pat. No. 102105179 teaches a device for the volatilization of essential oil. The device includes a container for receiving liquid fuel and a lid. The container also receives a wick. One end of the wick is to be immersed in the liquid fuel. The lid is disposed on the top of the container. The lid is provided with a perforation through which the wick is inserted. The lid has a body with a thickness and the top of the body is provided with an inner wall and an outer wall. A holding space for the essential oil is delimited between the inner and the outer walls. There is a first wall between the top of the inner wall and the bottom surface of the lid body. The lid has a first height measured between the top of the inner wall and the bottom of the body and a second height measured between the top of the outer wall and the bottom of the body. The first and the second heights are both greater than the thickness of the lid. The device can provide a flame for vaporizing the essential oil.

It is desirable that such device is adapted to vaporize various essential oils having different applicable temperature ranges and to transfer controlled heat for achieving a stable volatilization effect. However, it is not easy to control the heat, especially if the flame is influenced by wind or when the ambient temperature changes. In a windy environment, if the essential oil is in a volatile state, it may be ignited easily due to the fluttering of the flame, thereby causing danger. Further, it is inconvenient to add fuel into the device. When adding fuel, the lid has to be removed before adding the fuel, but the essential oil is contained in the lid. Also, the essential oil may contact the wick or flow into the fuel below in the process of adding essential oil or moving the device, thereby causing fuel pollution and incomplete combustion. Further, the heat cannot be controlled stably when it is conducted. Further, when the essential oil is vaporized, the temperature of the device can continue to rise, which may burn the user.

Apart from that, it is desirable that such device produces a flame which is aesthetic.

The present invention is, therefore, intended to obviate or at least alleviate the problems encountered in the prior art.

### SUMMARY OF THE INVENTION

According to the present invention, a combustion device adapted to the diffusion of fragrance includes a main body with an internal space divided into a heating zone and a fuel zone which provides heat to the heating zone. Moreover, the main body defines a ventilation hole through which air passes and circulates in the main body. The heating zone is adjacent to the top of the main body. The fuel zone is adjacent to the bottom of the main body. The fuel zone receives a wick. The ventilation hole extends vertically to the heating zone. The combustion device also includes a fragrance diffuser diffusing fragrance disposed in the heating zone. The fragrance diffuser includes an insulator and a fragrance holder housed by the insulator. The insulator defines a hollow above the wick and a groove. The fragrance holder is disposed in the groove

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the public generally, and especially the scientists, engineers and practitioners in the art who are not familiar with patent or legal terms or phraseology, to determine quickly from a cursory inspection the nature and essence of the technical disclosure. The abstract is neither intended to define the invention, which is measured by the claims, nor is it intended to be limiting as to the scope of the invention in any way.

Other objectives, advantages, and new features of the present invention will become apparent from the following detailed description of the invention when considered in conjunction with the accompanied drawings.

### BRIEF DESCRIPTION OF THE DRAWINGS

FIG. 1 is a perspective view of a combustion device adapted to the diffusion of fragrance in accordance with the present invention.

FIG. 2 is an exploded perspective view of FIG. 1.

FIG. 3 is a cross-sectional view of FIG. 1.

FIG. 4 illustrates the use of the combustion device, with arrows indicating fragrance.

### DETAILED DESCRIPTION OF THE INVENTION

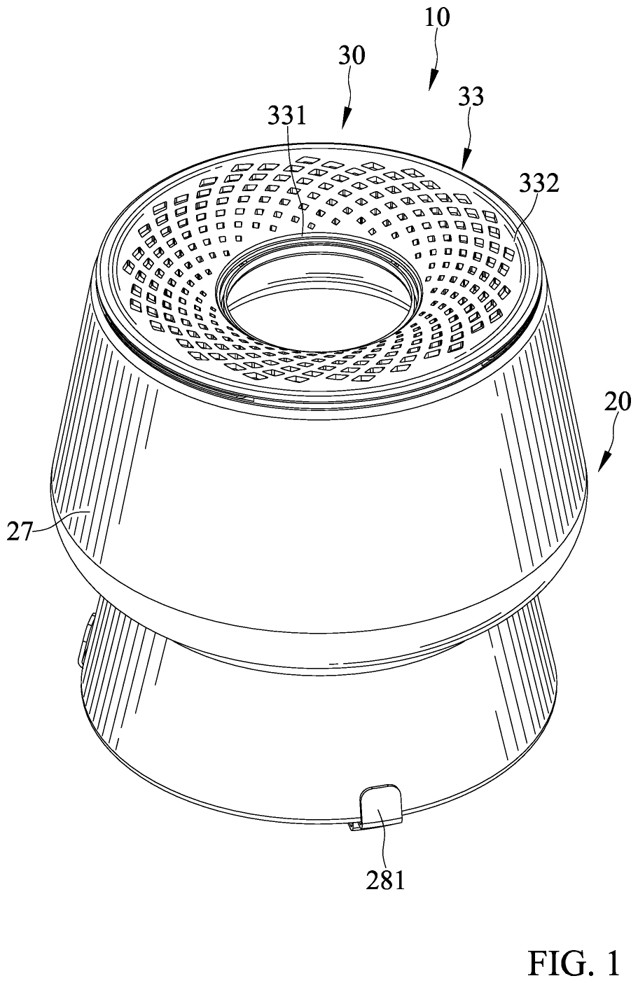

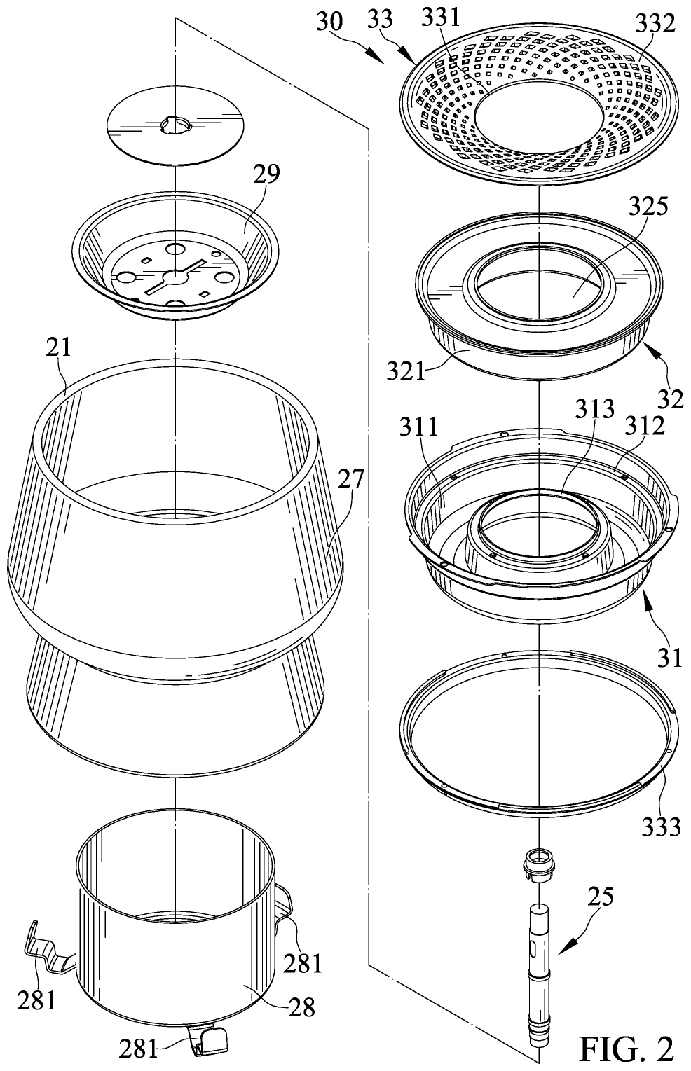

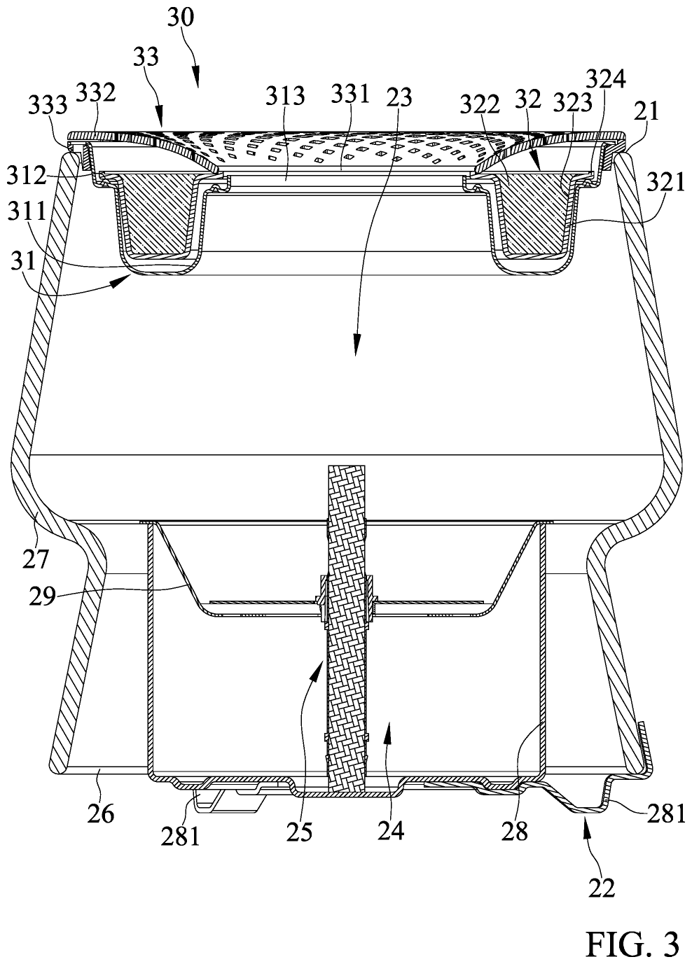

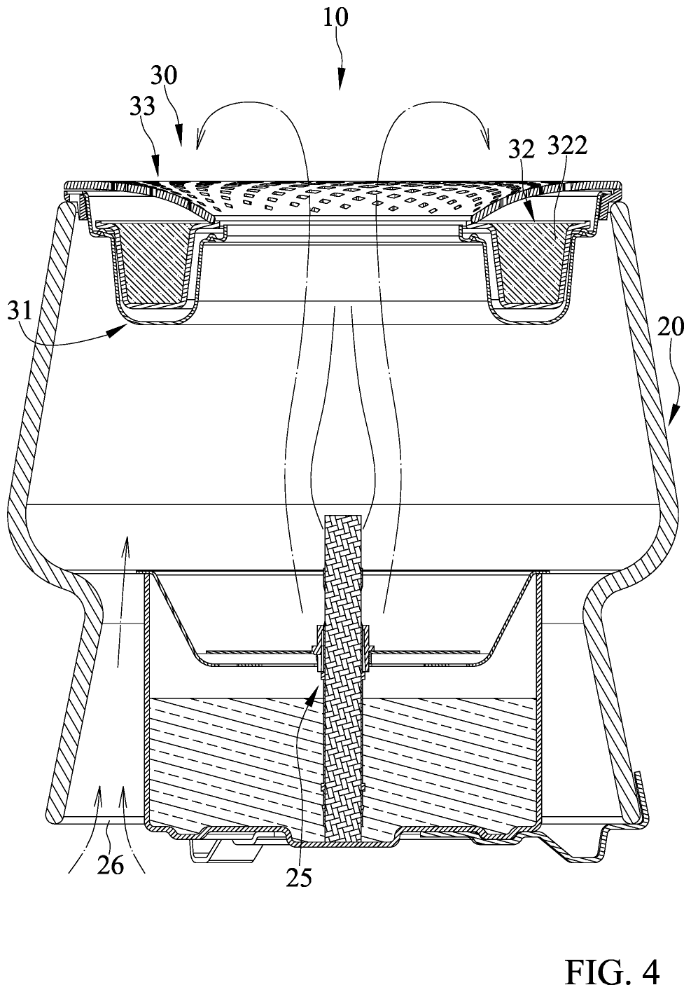

FIGS. 1 through 4 show a combustion device 10 adapted to the diffusion of fragrance in accordance with the present invention. The combustion device 10 has a main body 20 and a fragrance diffuser 30 diffusing fragrance.

The main body 20 has an internal space. The main body 20 has an outer body 27 delimiting the internal space. The internal space is divided into a heating zone 23 and a fuel zone 24. The fuel zone 24 provides heat to the heating zone 23. The heating zone 23 is adjacent to the top 21 of the main body 20. The fuel zone 24 is adjacent to the bottom 22 of the main body 20. The fuel zone 24 includes a fuel holder 28 with a reservoir for holding fuel. The fuel zone 24 receives a wick 25. The wick 25 has a first end inserted in the fuel holder 28 for drawing fuel therein. The fuel holder 28 is free of contact with the outer body 27.

The fuel holder 28 has at least one leg 281 and is supported on a surface by the at least one leg 281. The outer body 27 is releasably disposed on the at least one leg 281 and includes the bottom thereof spaced from the surface where the at least one leg 281 stands.

The fuel holder 28 includes a fuel cap 29 disposed thereon and enclosing the reservoir. The wick 25 has a second end inserted through the fuel holder 28. The fuel cap 29 is free of contact with the outer body 27. The wick 25 is made of metal and in the form of a column.

The internal space defines a ventilation hole 26 through which air passes and circulates in the main body 20, i.e., the internal space. The ventilation hole 26 extends vertically to the heating zone 23. The ventilation hole 26 extends through the bottom of the main body 20. The ventilation hole 26 is between an inner wall of the outer body 27 and an outer wall of the fuel holder 28.

The fragrance diffuser 30 is disposed in the heating zone 23. The fragrance diffuser 30 includes an insulator 31 and a fragrance holder 32 housed by the insulator 31.

The insulator 31 defines a hollow 313 above the wick 25 and a groove 311. The groove 311 has a closed bottom wall without any holes extending therethrough. The groove 311 has lateral walls which includes a first lateral wall extending from the bottom wall and delimiting a circumference of the hollow 313. The insulator 31 is made of metal.

The fragrance holder 32 is disposed in the groove 311. The fragrance holder 32 has a container 321 with a receptacle 323 for holding fragrance 322. The container 321 is disposed in the groove 311 and has a rim 324 rested on a shoulder 312 of the insulator 31. The shoulder 312 protrudes radially from a second lateral wall of the groove 311 which faces the first lateral wall oppositely. The container 321 is free of contact with the first lateral wall and the bottom wall. The container 321 defines a through hole 325 which opens to the hollow 313.

The fragrance diffuser 30 also includes a cover 33 disposed on the container 321 and enclosing the receptacle 323. The cover 33 is releasably attached to the insulator 31. The cover 33 defines a through hole 331 corresponding to the through hole 325. Specifically, a clip 333 is mounted on the insulator 31 and releasably fastening a body 332 of the cover 33. The clip 333 is in a form of a ring and put around the insulator 31.

In view of the foregoing, the combustion device 10 include the outer body 27 releasably disposed on the at least one leg 281 for allowing a user to fill fuel holder 28 conveniently. Further, the outer body 27 safeguards the flame of the wick 25 from becoming fluttered. Further, the fragrance diffuser 30 is prevented from burning the user.

The foregoing is merely illustrative of the principles of this invention and various modifications can be made by those skilled in the art without departing from the scope and spirit of the invention.

What is claimed is:

1. A combustion device adapted to the diffusion of fragrance comprising:
a main body with an internal space divided into a heating zone and a fuel zone which provides heat to the heating zone, and defining a ventilation hole through which air passes and circulates in the main body, wherein the heating zone is adjacent to the top of the main body, wherein the fuel zone is adjacent to the bottom of the main body, wherein the fuel zone receives a wick, and wherein the ventilation hole extends vertically to the heating zone; and
a fragrance diffuser diffusing fragrance disposed in the heating zone, wherein the fragrance diffuser includes an insulator and a fragrance holder housed by the insulator, wherein the insulator defines a hollow above the wick and a groove, and wherein the fragrance holder is disposed in the groove.

2. The combustion device as claimed in claim 1, wherein the ventilation hole extends through the bottom of the main body.

3. The combustion device as claimed in claim 1, wherein the groove has a closed bottom wall without any holes extending therethrough.

4. The combustion device as claimed in claim 3, wherein the groove has lateral walls which includes a first lateral wall extending from the bottom wall and delimiting a circumference of the hollow.

5. The combustion device as claimed in claim 4, wherein the fragrance holder has a container with a receptacle for holding fragrance, wherein the container is disposed in the groove and has a rim rested on a shoulder of the insulator, and wherein the container is free of contact with the first lateral wall and the bottom wall.

6. The combustion device as claimed in claim 5, wherein the shoulder protrudes radially from a second lateral wall of the groove which faces the first lateral wall oppositely.

7. The combustion device as claimed in claim 5, wherein the container defines a first through hole which opens to the hollow.

8. The combustion device as claimed in claim 7, wherein the fragrance diffuser includes a cover disposed on the container and enclosing the receptacle, wherein the cover is releasably attached to the insulator, and wherein the cover defines a second through hole corresponding to the first through hole.

9. The combustion device as claimed in claim 8 further comprising a clip mounted on the insulator and releasably fastening a body of the cover.

10. The combustion device as claimed in claim 9, wherein the clip is in a form of a ring and put around the insulator.

11. The combustion device as claimed in claim 1, wherein the insulator is made of metal.

12. The combustion device as claimed in claim 1, wherein the main body has an outer body delimiting the internal space, wherein the fuel zone includes a fuel holder with a reservoir for holding fuel, wherein the wick has a first end inserted in the fuel holder for drawing fuel therein, and wherein the fuel holder is free of contact with the outer body.

13. The combustion device as claimed in claim 12, wherein the fuel holder includes a fuel cap disposed thereon and enclosing the reservoir, wherein the wick has a second end inserted through the fuel holder, and wherein the fuel cap is free of contact with the outer body.

14. The combustion device as claimed in claim 13, wherein the wick is made of metal and in the form of a column.

15. The combustion device as claimed in claim 12, wherein the fuel holder has at least one leg and is supported on a surface by the at least one leg, wherein the outer body is releasably disposed on the at least one leg and includes the bottom thereof spaced from the surface where the at least one leg stands, and wherein the ventilation hole is between an inner wall of the outer body and an outer wall of the fuel holder.

16. The combustion device as claimed in claim 5, wherein the main body has an outer body delimiting the internal space, wherein the fuel zone includes a fuel holder with a reservoir for holding fuel, wherein the wick has a first end inserted in the fuel holder for drawing fuel therein, and wherein the fuel holder is free of contact with the outer body.

17. The combustion device as claimed in claim 8, wherein the main body has an outer body delimiting the internal space, wherein the fuel zone includes a fuel holder with a reservoir for holding fuel, wherein the wick has a first end inserted in the fuel holder for drawing fuel therein, and wherein the fuel holder is free of contact with the outer body.

18. The combustion device as claimed in claim 16, wherein the insulator is made of metal.

19. The combustion device as claimed in claim 17, wherein the insulator is made of metal.

20. The combustion device as claimed in claim 13, wherein the fuel holder has at least one leg and is supported on a surface by the at least one leg, wherein the outer body is releasably disposed on the at least one leg and includes the bottom thereof spaced from the surface where the at least one leg stands, and wherein the ventilation hole is between an inner wall of the outer body and an outer wall of the fuel holder.
